SEQUENCE LISTING (3 sequences)

SEQ ID NO: 1 - DNA, 18 bp, Artificial Sequence
  Pst Adaptor 1 primer
cacgatggatccagtgca

SEQ ID NO: 2 - DNA, 16 bp, Artificial Sequence
  Pst Adaptor 2 primer
ctggatccatcgtgca

SEQ ID NO: 3 - DNA, 16 bp, Artificial Sequence
  PstI Primer
gatgcatccagtgcag

(19) United States
(12) Patent Application Publication
(10) Pub. No.: US 2007/0048768 A1
(43) Pub. Date: Mar. 1, 2007

(54) Methods for screening for gene specific hybridization polymorphisms (GSHPs) and their use in genetic mapping and marker development

(75) Inventors: John Salmeron, Research Triangle Park, NC (US); Tong Zhu, Research Triangle Park, NC (US)

Correspondence Address: Larry W. Stults, Ph.D.;Syngenta Biotechnology, Inc., 3054 Cornwallis Road, Research Triangle Park, NC, 27709, US

(73) Assignee: Syngenta Participations AG, Basel (CH)

(21) Appl. No.: 11/472,789

(22) Filed: Jun. 22, 2006

Related U.S. Application Data
(60) Provisional application No. 60/695,781, filed on Jun. 30, 2005.

(51) Int. Cl. C12Q 1/68; G06F 19/00; C12P 19/34

(52) U.S. Cl. 435/6; 435/91.2; 702/20

(57) ABSTRACT

A method for identification of gene specific hybridization polymorphisms (GSHPs) and their use is presented. The method involves the steps of a) global screening for hybridization polymorphisms using microarray; b) enzyme mediated genome complexity reduction; c) enzyme mediated differential signal amplification and noise reduction; d) data extraction and GSHP identification; and e) use of GSHPs in high throughput screening.

### FIELD OF THE INVENTION

The present invention relates to the field of biotechnology. More specifically, the present invention relates to methods for screening for gene specific hybridization polymorphisms, for discovery of various types of such polymorphisms, and to the discovered polymorphisms and their use in marker development, for genetic mapping and marker assisted selection/breeding and genetic identification.

### BACKGROUND OF THE INVENTION

The development of molecular genetic markers has facilitated mapping and selection of agriculturally important traits in crop plants, and for the identification of genes associated with disease states or for personal identification in humans. Markers tightly linked to genes are an asset in the rapid identification of plant lines or of human individuals on the basis of genotype, as well as in plant breeding by the use of marker assisted selection (MAS). Introgressing particular genes into a desired crop line or cultivar would also be facilitated by using suitable DNA markers.

Molecular Markers and Marker Assisted Selection

A genetic map is a graphical representation of a genome (or a portion of a genome such as a single chromosome) where the distances between landmarks on the chromosome are measured by the recombination frequencies between the landmarks. A genetic landmark can be any of a variety of known polymorphic markers, for example but not limited to, molecular markers such as SSR markers, RFLP markers, or SNP markers. Furthermore, SSR markers can be derived from genomic or expressed nucleic acids (e.g., ESTs). The nature of these physical landmarks and the methods used to detect them vary, but all of these markers are physically distinguishable from each other (as well as from the plurality of alleles of any one particular marker) on the basis of polynucleotide length and/or sequence.

Although specific DNA sequences which encode proteins are generally well-conserved across a species, other regions of DNA (typically non-coding) tend to accumulate polymorphism, and therefore, can be variable between individuals of the same species. Such regions provide the basis for numerous molecular genetic markers. In general, any differentially inherited polymorphic trait (including nucleic acid polymorphism) that segregates among progeny is a potential marker. The genomic variability can be of any origin, for example, insertions, deletions, duplications, repetitive elements, point mutations, recombination events, or the presence and sequence of transposable elements. Molecular markers in many species, associated with numerous genes, are known in the art, and are published or available from various sources, such as the SOYBASE internet resource for markers in soybean. Similarly, numerous methods for detecting molecular markers are also well-established.

The primary motivation for developing molecular marker technologies from the point of view of plant breeders has been the possibility to increase breeding efficiency through marker assisted selection (MAS). A molecular marker allele that demonstrates linkage disequilibrium with a desired phenotypic trait (e.g., a quantitative trait locus, or QTL, for example, resistance to a particular disease) provides a useful tool for the selection of a desired trait in a plant population. The key components to the implementation of this approach are: (i) the creation of a dense genetic map of molecular markers, (ii) the detection of QTL based on statistical associations between marker and phenotypic variability, (iii) the definition of a set of desirable marker alleles based on the results of the QTL analysis, and (iv) the use and/or extrapolation of this information to the current set of breeding germplasm to enable marker-based selection decisions to be made.

Two types of markers are frequently used in marker assisted selection protocols, namely simple sequence repeat (SSR, also known as microsatellite) markers, and single nucleotide polymorphism (SNP) markers.

Molecular markers that rely on single nucleotide polymorphisms (SNPs) are well known in the art. Various techniques have been developed for the detection of SNPs, including allele specific hybridization (ASH; see, e.g., Coryell et al., (1999) “Allele specific hybridization markers for soybean,” Theor. Appl. Genet., 98:690-696). Additional types of molecular markers are also widely used, including but not limited to expressed sequence tags (ESTs) and SSR markers, restriction fragment length polymorphism (RFLP), amplified fragment length polymorphism (AFLP), randomly amplified polymorphic DNA (RAPD) and isozyme markers. A wide range of protocols are known to one of skill in the art for detecting this variability, and these protocols are frequently specific for the type of polymorphism they are designed to detect. For example, PCR amplification, single-strand conformation polymorphisms (SSCP) and self-sustained sequence replication (3SR; see Chan and Fox, “NASBA and other transcription-based amplification methods for research and diagnostic microbiology,” Reviews in Medical Microbiology 10:185-196 [1999]).

Linkage of one molecular marker to another molecular marker is measured as a recombination frequency. In general, the closer two loci (e.g., two SSR markers) are on the genetic map, the closer they lie to each other on the physical map. A relative genetic distance (determined by crossing over frequencies, measured in centimorgans; cM) is generally proportional to the physical distance (measured in base pairs, e.g., kilobase pairs [kb] or megabasepairs [Mbp]) that two linked loci are separated from each other on a chromosome. A lack of precise proportionality between cM and physical distance can result from variation in recombination frequencies for different chromosomal regions, e.g., some chromosomal regions are recombinational “hot spots,” while others regions do not show any recombination, or only demonstrate rare recombination events. In general, the closer one marker is to another marker, whether measured in terms of recombination or physical distance, the more strongly they are linked. In some aspects, the closer a molecular marker is to a gene that encodes a polypeptide that imparts a particular phenotype (drought tolerance, for example), whether measured in terms of recombination or physical distance, the better that marker serves to tag the desired phenotypic trait.

Genetic mapping variability can also be observed between different populations of the same crop species. In spite of this variability in the genetic map that may occur between populations, genetic map and marker information derived from one population generally remains useful across multiple populations in identification of plants with desired traits, counter-selection of plants with undesirable traits and in guiding MAS.

QTL Mapping

It is the goal of the plant breeder to select plants and enrich the plant population for individuals that have desired traits, for example, heat stress tolerance, leading ultimately to increased agricultural productivity. It has been recognized for quite some time that specific chromosomal loci (or intervals) can be mapped in an organism's genome that correlate with particular quantitative phenotypes. Such loci are termed quantitative trait loci, or QTL. The plant breeder can advantageously use molecular markers to identify desired individuals by identifying marker alleles that show a statistically significant probability of co-segregation with a desired phenotype (e.g., pathogenic infection tolerance), manifested as linkage disequilibrium. By identifying a molecular marker or clusters of molecular markers that co-segregate with a quantitative trait, the breeder is thus identifying a QTL. By identifying and selecting a marker allele (or desired alleles from multiple markers) that associates with the desired phenotype, the plant breeder is able to rapidly select a desired phenotype by selecting for the proper molecular marker allele (a process called marker-assisted selection, or MAS). The more molecular markers that are placed on the genetic map, the more potentially useful that map becomes for conducting MAS.

Multiple experimental paradigms have been developed to identify and analyze QTL (see, e.g., Jansen (1996) Trends Plant Sci 1:89). The majority of published reports on QTL mapping in crop species have been based on the use of the bi-parental cross (Lynch and Walsh (1997) Genetics and Analysis of Quantitative Traits, Sinauer Associates, Sunderland). Typically, these paradigms involve crossing one or more parental pairs, which can be, for example, a single pair derived from two inbred strains, or multiple related or unrelated parents of different inbred strains or lines, which each exhibit different characteristics relative to the phenotypic trait of interest. Typically, this experimental protocol involves deriving 100 to 300 segregating progeny from a single cross of two divergent inbred lines (e.g., selected to maximize phenotypic and molecular marker differences between the lines). The parents and segregating progeny are genotyped for multiple marker loci and evaluated for one to several quantitative traits (e.g., disease resistance, drought tolerance, fruit color, etc.). QTL are then identified as significant statistical associations between genotypic values and phenotypic variability among the segregating progeny. The strength of this experimental protocol comes from the utilization of the inbred cross, because the, resulting F1 parents all have the same linkage phase. Thus, after selfing of the F1 plants, all segregating progeny (F2) are informative and linkage disequilibrium is maximized, the linkage phase is known, there are only two QTL alleles, and, except for backcross progeny, the frequency of each QTL allele is 0.5.

Numerous statistical methods for determining whether markers are genetically linked to a QTL (or to another marker) are known to those of skill in the art and include, e.g., standard linear models, such as ANOVA or regression mapping (Haley and Knott (1992) Heredity 69:315), maximum likelihood methods such as expectation-maximization algorithms, (e.g., Lander and Botstein (1989) “Mapping Mendelian factors underlying quantitative traits using RFLP linkage maps,” Genetics 121:185-199; Jansen (1992) “A general mixture model for mapping quantitative trait loci by using molecular markers,” Theor. Appl. Genet., 85:252-260; Jansen (1993) “Maximum likelihood in a generalized linear finite mixture model by using the EM algorithm,” Biometrics 49:227-231; Jansen (1994) “Mapping of quantitative trait loci by using genetic markers: an overview of biometrical models,” In J. W. van Ooijen and J. Jansen (eds.), Biometrics in Plant breeding: applications of molecular markers, pp. 116-124, CPRO-DLO Netherlands; Jansen (1996) “A general Monte Carlo method for mapping multiple quantitative trait loci,” Genetics 142:305-311; and Jansen and Stam (1994) “High Resolution of quantitative trait into multiple loci via interval mapping,” Genetics 136:1447-1455). Exemplary statistical methods include single point marker analysis, interval mapping (Lander and Botstein (1989) Genetics 121:185), composite interval mapping, penalized regression analysis, complex pedigree analysis, MCMC analysis, MQM analysis (Jansen (1994) Genetics 138:871), HAPLO-IM+ analysis, HAPLO-MQM analysis, and HAPLO-MQM+ analysis, Bayesian MCMC, ridge regression, identity-by-descent analysis, Haseman-Elston regression, any of which are suitable in the context of the present invention. In addition, additional details regarding alternative statistical methods applicable to complex breeding populations which can be used to identify and localize QTLs are described in: U.S. Ser. No. 09/216,089 by Beavis et al. “QTL MAPPING IN PLANT BREEDING POPULATIONS” and PCT/US00/34971 by Jansen et al. “MQM MAPPING USING HAPLOTYPED PUTATIVE QTLS ALLELES: A SIMPLE APPROACH FOR MAPPING QTLS IN PLANT BREEDING POPULATIONS.” Any of these approaches are computationally intensive and are usually performed with the assistance of a computer based system and specialized software. Appropriate statistical packages are available from a variety of public and commercial sources, and are known to those of skill in the art.

### SUMMARY OF THE INVENTION

A high-throughput method to screen for gene specific hybridization polymorphisms in any genome, including and particularly in complex genomes, was developed. Gene specific hybridization polymorphisms are anonymous polymorphisms discovered in the coding region of targeted genes. The invented method can detect single nucleotide polymorphism (SNP), and associated restriction fragment length polymorphism (RFLP), amplified fragment length polymorphism (AFLP), and secondary structural polymorphism simultaneously (FIG. 1). The detected polymorphism can be used directly as hybridization marker in high-throughput screening, or transformed to SNPs, and develop into a functional polymorphism marker or use as marker using non-hybridization based readout technologies. Such markers can be used in plant breeding applications for marker assisted selection/breeding, or in plant or animal/human applications for identification of genotypes, identification of quantitative trait loci, and/or for gene mapping applications.

The present method includes the following general components: 1) global genomic screening for hybridization polymorphism using microarray by comparative genomic hybridization; 2) enzyme mediated genome complexity reduction; 3) enzyme mediated differential signal amplification and noise reduction; 4) data extraction and GSHP identification; and 5) use of GSHP in high throughput screening. Among these components, enzyme mediated genome complexity reduction and enzyme mediated differential signal amplification and noise reduction are particularly useful for screening the genomes of organisms with complex genomes. For those organisms with simple genomes, these components are optional and can be substitute with direct incorporation of fluorescent labels using methods such as random hexamer labeling.

The invention provides a method for detection of gene specific hybridization polymorphisms in polynucleotide sequences of genomic DNA, the method comprising:
a selecting short oligonucleotide sequences complementary to the genomic polynucleotide sequences, said short oligonucleotide sequences to be synthesized directly onto or synthesized and placed onto a microarray surface;
b. preparing genomic DNA from two genetic sources and subjecting said genomic DNA to site-specific restriction using one or more restriction enzymes to produce restriction fragment length polymorphisms (RFLPs);
c. selectively amplifying RFLPs of a selected size range to create amplified polymorphism targets;
d. fragmenting the amplified targets randomly into fragments of from about 50 to about 200 bases and end-labeling the fragments unselectively;
e. hybridizing the end-labeled fragments to the short oligonucleotide sequences on the microarray surface; and
f. quantifying the signals from the hybridization and detecting polymorphisms.

The present method can be further used to detect GSHP in phylogenetically closely related species A and B using a microarray with probes from the model species B. To do this, the sequence similarity between the species A and B should be assessed computationally and/or experimentally. If a computational approach is used, the representative sequences from A should be BLAST against B. If an experimental approach is used, genomic DNA from species A should be extracted, labeled, and cross hybridized to the microarray with probes designed from the species B. If the number of similar sequences is above the acceptable threshold, then the genomics DNA of species A could be used in a similar fashion as native genomic DNA B for GSHP detection within the homologous sequences.

The invention provides a cost-effective assay for scan gene polymorphisms at whole genome scale, and provides numerous advantages, as outlined below.

Compared to conventional mapping methods, the present invention provides:
Genome-wide coverage—Although they only represent 0.7-1% of the genome sequences, the probe sequences usually cover 60-80% of the genes in the genome.
The ability to generate large numbers of markers for ultra-high density genome mapping. It is estimated that the average polymorphism rate in maize coding sequences to be 1 in 124 bases. One experiment could screen up to 3.25×10⁵ polymorphisms. Even if only one base among the 25 bases of the probe is considered to be sensitive to the detection, it could at least identify 1.3×10⁴ polymorphisms. These potential markers could be used for marker-assisted selection, and for generating an ultra-high density genetic map.
All markers are gene markers—gene markers could influence or be responsible to the complex traits. GeneChip microarrays were designed based on gene or EST sequences. Thus the identified polymorphisms will be associated with the genes. Compared to random markers used for mapping, the gene markers could be biologically functional and could thus facilitate the functional analysis for trait dissection.
Convertible to high-throughput compatible form—the oligonucleotide probes containing polymorphism markers could be converted to SNP markers by sequencing, as 80-90% of the SFPs (single feature polymorphisms) are SNPs. The SFP markers could also be directly utilized since they could be easily migrated from the regular GeneChip to a low cost mini-marker GeneChip. This enables the utility of the markers for low-cost, high-throughput screening.
Fast—one GeneChip experiment could survey up to 3.25×10⁷ bases for maize and 5.75×10⁶ bases for tomato, and it only takes two days. An entire mapping project can be done in 4-6 months
Cost effective—it is cost-effective. Based on $500 cost for chip and reagents, it is estimated the per marker discovery cost to be 0.25 cent in maize

Compared to other microarray-based mapping methods:
Low cost for marker discovery—other microarray-based methods such as tilling GeneChip arrays have high cost
Applicable to organisms with a complex genome, including most of the higher organisms, such as crop species, animals, and ecological model systems
Accurate and precise—it minimizes the interference from the non-specific binding
Focused on gene markers—methylation filtering enriches the gene fragments in the labeled targets
It increases the labeling efficiency by reducing the complexity of the target pool.
It increases signal intensity and differential signals by preferential amplification of the targets
It detects only genetic variations not the transcription variations which could be greatly affected by the environments and experimental conditions.

The present method can be applied to the following non-limiting applications which have been widely used in agricultural and medical science and practice: 1) construction of ultra-high density gene map; 2) identify markers for single gene traits or QTLs by bulk segregant analysis (BSA) and similar approaches; 3) associate QTL and candidate genes through whole genome linkage analyses or association studies; and 4) high throughput screening using diagnostic marker.

### BRIEF DESCRIPTION OF THE DRAWINGS

FIG. 1. Detection of sequence polymorphism by target-probe hybridization. The dark and light lines represent the target sequences from different genetic varieties that are homologous to the detection probe. The circles represent the sequence polymorphism between the varieties.

FIG. 2. Experimental procedure for genome complexity reduction

FIG. 3. Comparison of frequency of probes with different signal intensities in soybean (native detection) and common beans (heterologous detection) using a soybean GeneChip array. It is clear that a significant number of soybean probes can cross hybridize common bean targets.

### DETAILED DESCRIPTION OF THE INVENTION

Definitions

Before describing the present invention in detail, it is to be understood that this invention is not limited to particular embodiments, which can, of course, vary. It is also to be understood that the terminology used herein is for the purpose of describing particular embodiments only, and is not intended to be limiting. As used in this specification and the appended claims, terms in the singular and the singular forms “a,” “an” and “the,” for example, include plural referents unless the content clearly dictates otherwise. Thus, for example, reference to “plant,” “the plant” or “a plant” also includes a plurality of plants; also, depending on the context, use of the term “plant” can also include genetically similar or identical progeny of that plant; use of the term “a nucleic acid” optionally includes, as a practical matter, many copies of that nucleic acid molecule; similarly, the term “probe” optionally (and typically) encompasses many similar or identical probe molecules.

Unless otherwise indicated, nucleic acids are written left to right in 5′ to 3′ orientation. Numeric ranges recited within the specification are inclusive of the numbers defining the range and include each integer or any non-integer fraction within the defined range. Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used in accordance with the definitions set out below.

A “plant” can be a whole plant, any part thereof, or a cell or tissue culture derived from a plant. Thus, the term “plant” can refer to any of: whole plants, plant components or organs (e.g., leaves, stems, roots, etc.), plant tissues, seeds, plant cells, and/or progeny of the same. A plant cell is a cell of a plant, taken from a plant, or derived through culture from a cell taken from a plant. Thus, the term “corn plant” includes whole corn plants, corn plant cells, corn plant protoplast, corn plant cell or corn tissue culture from which corn plants can be regenerated, corn plant calli, corn plant clumps and corn plant cells that are intact in corn plants or parts of corn plants, such as corn seeds, corn pods, corn flowers, corn cotyledons, corn leaves, corn stems, corn buds, corn roots, corn root tips and the like.

“Germplasm” refers to genetic material of or from an individual (e.g., a plant), a group of individuals (e.g., a plant line, variety or family), or a clone derived from a line, variety, species, or culture. The germplasm can be part of an organism or cell, or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants may be grown, or plant parts, such as leafs, stems, pollen, or cells, which can be cultured into a whole plant.

The term “allele” refers to one of two or more different nucleotide sequences that occur at a specific locus. For example, a first allele can occur on one chromosome, while a second allele occurs on a second homologous chromosome, e.g., as occurs for different chromosomes of a heterozygous individual, or between different homozygous or heterozygous individuals in a population. A “favorable allele” is the allele at a particular locus that confers, or contributes to, an agronomically desirable phenotype, e.g., tolerance to a pest or to drought, or alternatively, is an allele that allows the identification of susceptible plants that can be removed from a breeding program or planting. A favorable allele of a marker is a marker allele that segregates with the favorable phenotype, or alternatively, segregates with susceptible plant phenotype, therefore providing the benefit of identifying drought-prone plants. A favorable allelic form of a chromosome segment is a chromosome segment that includes a nucleotide sequence that contributes to superior agronomic performance at one or more genetic loci physically located on the chromosome segment. “Allele frequency” refers to the frequency (proportion or percentage) at which an allele is present at a locus within an individual, within a line, or within a population of lines. For example, for an allele “A,” diploid individuals of genotype “AA,” “Aa,” or “aa” have allele frequencies of 1.0, 0.5, or 0.0, respectively. One can estimate the allele frequency within a line by averaging the allele frequencies of a sample of individuals from that line. Similarly, one can calculate the allele frequency within a population of lines by averaging the allele frequencies of lines that make up the population. For a population with a finite number of individuals or lines, an allele frequency can be expressed as a count of individuals or lines (or any other specified grouping) containing the allele.

An allele “positively” correlates with a trait when it is linked to it and when presence of the allele is an indictor that the desired trait or trait form will occur in a plant comprising the allele. An allele negatively correlates with a trait when it is linked to it and when presence of the allele is an indicator that a desired trait or trait form will not occur in a plant comprising the allele.

An individual is “homozygous” if the individual has only one type of allele at a given locus (e.g., a diploid individual has a copy of the same allele at a locus for each of two homologous chromosomes). An individual is “heterozygous” if more than one allele type is present at a given locus (e.g., a diploid individual with one copy each of two different alleles). The term “homogeneity” indicates that members of a group have the same genotype at one or more specific loci. In contrast, the term “heterogeneity” is used to indicate that individuals within the group differ in genotype at one or more specific loci.

A “locus” is a chromosomal region where a polymorphic nucleic acid, trait determinant, gene or marker is located. Thus, for example, a “gene locus” is a specific chromosome location in the genome of a species where a specific gene can be found.

The term “quantitative trait locus” or “QTL” refers to a polymorphic genetic locus with at least two alleles that differentially affect the expression of a phenotypic trait in at least one genetic background, e.g., in at least one breeding population or progeny. QTL are typically identified or “marked” using molecular markers.

The terms “marker,” “molecular marker,” “marker nucleic acid,” and “marker locus” refer to a nucleotide sequence or encoded product thereof (e.g., a protein) used as a point of reference when identifying a linked locus. A marker can be derived from genomic nucleotide sequence or from expressed nucleotide sequences (e.g., from a spliced RNA, a cDNA, etc.), or from an encoded polypeptide. The term also refers to nucleic acid sequences complementary to or flanking the marker sequences, such as nucleic acids used as probes or primer pairs capable of amplifying the marker sequence. A “marker probe” is a nucleic acid sequence or molecule that can be used to identify the presence of a marker locus, e.g., a nucleic acid probe that is complementary to a marker locus sequence. Alternatively, in some aspects, a marker probe refers to a probe of any type that is able to distinguish (i.e., genotype) the particular allele that is present at a marker locus. Nucleic acids are “complementary” when they specifically hybridize in solution, e.g., according to Watson-Crick base pairing rules. A “marker locus” is a locus that can be used to track the presence of a second linked locus, e.g., a linked locus that encodes or contributes to expression of a phenotypic trait. For example, a marker locus can be used to monitor segregation of alleles at a locus, such as a QTL, that are genetically or physically linked to the marker locus. Thus, a “marker allele,” alternatively an “allele of a marker locus” is one of a plurality of polymorphic nucleotide sequences found at a marker locus in a population that is polymorphic for the marker locus. Each of the identified markers is expected to be in close physical and genetic proximity (resulting in physical and/or genetic linkage) to a genetic element, e.g., a QTL, which contributes to tolerance.

“Genetic markers” are nucleic acids that are polymorphic in a population and where the alleles of which can be detected and distinguished by one or more analytic methods, e.g., RFLP, AFLP, isozyme, SNP, SSR, and the like. The terms “genetic marker” and “molecular marker” refer to a genetic locus (a “marker locus”) that can be used as a point of reference when identifying a genetically linked locus such as a QTL. Such a marker is also referred to as a QTL marker. The term also refers to nucleic acid sequences complementary to the genomic sequences, such as nucleic acids used as probes.

Markers corresponding to genetic polymorphisms between members of a population can be detected by methods well-established in the art. These include, e.g., PCR-based sequence specific amplification methods, detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of single nucleotide polymorphisms (SNPs), or detection of amplified fragment length polymorphisms (AFLPs). Well established methods are also know for the detection of expressed sequence tags (ESTs) and SSR markers derived from EST sequences and randomly amplified polymorphic DNA (RAPD).

A “genetic map” is a description of genetic linkage relationships among loci on one or more chromosomes (or linkage groups) within a given species, generally depicted in a diagrammatic or tabular form. “Genetic mapping” is the process of defining the linkage relationships of loci through the use of genetic markers, populations segregating for the markers, and standard genetic principles of recombination frequency. A “genetic map location” is a location on a genetic map relative to surrounding genetic markers on the same linkage group where a specified marker can be found within a given species. In contrast, a physical map of the genome refers to absolute distances (for example, measured in base pairs or isolated and overlapping contiguous genetic fragments, e.g., contigs). A physical map of the genome does not take into account the genetic behavior (e.g., recombination frequencies) between different points on the physical map.

A “genetic recombination frequency” is the frequency of a crossing over event (recombination) between two genetic loci. Recombination frequency can be observed by following the segregation of markers and/or traits following meiosis. A genetic recombination frequency can be expressed in centimorgans (cM), where one cM is the distance between two genetic markers that show a 1% recombination frequency (i.e., a crossing-over event occurs between those two markers once in every 100 cell divisions).

As used herein, the term “linkage” is used to describe the degree with which one marker locus is “associated with” another marker locus or some other locus (for example, a tolerance locus).

As used herein, linkage equilibrium describes a situation where two markers independently segregate, i.e., sort among progeny randomly. Markers that show linkage equilibrium are considered unlinked (whether or not they lie on the same chromosome).

As used herein, linkage disequilibrium describes a situation where two markers segregate in a non-random manner, i.e., have a recombination frequency of less than 50% (and by definition, are separated by less than 50 cM on the same linkage group). Markers that show linkage disequilibrium are considered linked. Linkage occurs when the marker locus and a linked locus are found together in progeny plants more frequently than not together in the progeny plants. As used herein, linkage can be between two markers, or alternatively between a marker and a phenotype. A marker locus can be associated with (linked to) a trait, e.g., a marker locus can be associated with tolerance or improved tolerance to a plant pathogen when the marker locus is in linkage disequilibrium with the tolerance trait. The degree of linkage of a molecular marker to a phenotypic trait (e.g., a QTL) is measured, e.g., as a statistical probability of co-segregation of that molecular marker with the phenotype.

As used herein, the linkage relationship between a molecular marker and a phenotype is given as a “probability” or “adjusted probability.” The probability value is the statistical likelihood that the particular combination of a phenotype and the presence or absence of a particular marker allele is random. Thus, the lower the probability score, the greater the likelihood that a phenotype and a particular marker will co-segregate. In some aspects, the probability score is considered “significant” or “nonsignificant.” In some embodiments, a probability score of 0.05 (p=0.05, or a 5% probability) of random assortment is considered a significant indication of co-segregation. However, the present invention is not limited to this particular standard, and an acceptable probability can be any probability of less than 50% (p=0.5). For example, a significant probability can be less than 0.25, less than 0.20, less than 0.15, or less than 0.1.

The term “linkage disequilibrium” refers to a non-random segregation of genetic loci or traits (or both). In either case, linkage disequilibrium implies that the relevant loci are within sufficient physical proximity along a length of a chromosome so that they segregate together with greater than random (i.e., non-random) frequency (in the case of co-segregating traits, the loci that underlie the traits are in sufficient proximity to each other). Linked loci co-segregate more than 50% of the time, e.g., from about 51% to about 100% of the time. The term “physically linked” is sometimes used to indicate that two loci, e.g., two marker loci, are physically present on the same chromosome.

Advantageously, the two linked loci are located in close proximity such that recombination between homologous chromosome pairs does not occur between the two loci during meiosis with high frequency, e.g., such that linked loci co-segregate at least about 90% of the time, e.g., 91%,92%, 93%,94%, 95%, 96%, 97%, 98%, 99%, 99.5%, 99.75%, or more of the time.

The phrase “closely linked,” in the present application, means that recombination between two linked loci occurs with a frequency of equal to or less than about 10% (i.e., are separated on a genetic map by not more than 10 cM). Put another way, the closely linked loci co-segregate at least 90% of the time. Marker loci are especially useful in the present invention when they demonstrate a significant probability of co-segregation (linkage) with a desired trait (e.g., pathogenic tolerance). For example, in some aspects, these markers can be termed linked QTL markers. In other aspects, especially useful molecular markers are those markers that are linked or closely linked to QTL markers.

In some aspects, linkage can be expressed as any desired limit or range. For example, in some embodiments, two linked loci are two loci that are separated by less than 50 cM map units. In other embodiments, linked loci are two loci that are separated by less than 40 cM. In other embodiments, two linked loci are two loci that are separated by less than 30 cM. In other embodiments, two linked loci are two loci that are separated by less than 25 cM. In other embodiments, two linked loci are two loci that are separated by less than 20 cM. In other embodiments, two linked loci are two loci that are separated by less than 15 cM. In some aspects, it is advantageous to define a bracketed range of linkage, for example, between 10 and 20 cM, or between 10 and 30 cM, or between 10 and 40 cM.

The more closely a marker is linked to a second locus, the better an indicator for the second locus that marker becomes. Thus, in one embodiment, closely linked loci such as a marker locus and a second locus (e.g., a QTL marker) display an inter-locus recombination frequency of 10% or less, preferably about 9% or less, still more preferably about 8% or less, yet more preferably about 7% or less, still more preferably about 6% or less, yet more preferably about 5% or less, still more preferably about 4% or less, yet more preferably about 3% or less, and still more preferably about 2% or less. In highly preferred embodiments, the relevant loci (e.g., a marker locus and a QTL marker) display a recombination a frequency of about 1% or less, e.g., about 0.75% or less, more preferably about 0.5% or less, or yet more preferably about 0.25% or less. Two loci that are localized to the same chromosome, and at such a distance that recombination between the two loci occurs at a frequency of less than 10% (e.g., about 9%, 8%, 7%, 6%, 5%, 4%, 3%, 2%, 1%, 0.75%, 0.5%, 0.25%, or less) are also said to be “proximal to” each other. In some cases, two different markers can have the same genetic map coordinates. In that case, the two markers are in such close proximity to each other that recombination occurs between them with such low frequency that it is undetectable.

When referring to the relationship between two genetic elements, such as a genetic element contributing to tolerance and a proximal marker, “coupling” phase linkage indicates the state where the “favorable” allele at the tolerance locus is physically associated on the same chromosome strand as the “favorable” allele of the respective linked marker locus. In coupling phase, both favorable alleles are inherited together by progeny that inherit that chromosome strand. In “repulsion” phase linkage, the “favorable” allele at the locus of interest (e.g., a QTL for tolerance) is physically linked with an “unfavorable” allele at the proximal marker locus, and the two “favorable” alleles are not inherited together (i.e., the two loci are “out of phase” with each other).

As used herein, the terms “chromosome interval” or “chromosome segment” designate a contiguous linear span of genomic DNA that resides in planta on a single chromosome. The genetic elements or genes located on a single chromosome interval are physically linked. The size of a chromosome interval is not particularly limited.

In some aspects, for example in the context of the present invention, generally the genetic elements located within a single chromosome interval are also genetically linked, typically within a genetic recombination distance of, for example, less than or equal to 20 centimorgan (cM), or alternatively, less than or equal to 10 cM. That is, two genetic elements within a single chromosome interval undergo recombination at a frequency of less than or equal to 20% or 10%

In one aspect, any marker of the invention is linked (genetically and physically) to any other marker that is at or less than 50 cM distant. In another aspect, any marker of the invention is closely linked (genetically and physically) to any other marker that is in close proximity, e.g., at or less than 10 cM distant. Two closely linked markers on the same chromosome can be positioned 9, 8, 7, 6, 5, 4, 3, 2, 1, 0.75, 0.5 or 0.25 cM or less from each other.

“Tolerance” or “improved tolerance” in a plant to biotic or abiotic stress is an indication that the plant is less affected with respect to yield and/or survivability or other relevant agronomic measures, upon occurrence of the stress, than a less tolerant or more “susceptible” plant. Tolerance is a relative term, indicating that the affected plant produces better yield than another similarly affected, more susceptible plant. That is, the stress causes a reduced decrease in survival and/or yield in a tolerant plant, as compared to a susceptible plant. One of skill will appreciate that plant tolerance to various stresses varies widely, and that tolerance also will vary depending on the severity of the stress. However, by simple observation, one of skill can determine the relative tolerance or susceptibility of different plants, plant lines or plant families to stress of a given severity.

The term “crossed” or “cross” in the context of this invention means the fusion of gametes via pollination to produce progeny (e.g., cells, seeds or plants). The term encompasses both sexual crosses (the pollination of one plant by another) and selfing (self-pollination, e.g., when the pollen and ovule are from the same plant).

The term “introgression” refers to the transmission of a desired allele of a genetic locus from one genetic background to another. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., a selected allele of a marker, a QTL, a transgene, or the like. In any case, offspring comprising the desired allele can be repeatedly backcrossed to a line having a desired genetic background and selected for the desired allele, to result in the allele becoming fixed in a selected genetic background.

A “line” or “strain” is a group of individuals of identical parentage that are generally inbred to some degree and that are generally homozygous and homogeneous at most loci (isogenic or near isogenic). A “subline” refers to an inbred subset of descendents that are genetically distinct from other similarly inbred subsets descended from the same progenitor. Traditionally, a “subline” has been derived by inbreeding the seed from an individual soybean plant selected at the F3 to F5 generation until the residual segregating loci are “fixed” or homozygous across most or all loci. Commercial soybean varieties (or lines) are typically produced by aggregating (“bulking”) the self-pollinated progeny of a single F3 to F5 plant from a controlled cross between 2 genetically different parents. While the variety typically appears uniform, the self-pollinating variety derived from the selected plant eventually (e.g., F8) becomes a mixture of homozygous plants that can vary in genotype at any locus that was heterozygous in the originally selected F3 to F5 plant. In the context of the invention, marker-based sublines, that differ from each other based on qualitative polymorphism at the DNA level at one or more specific marker loci, are derived by genotyping a sample of seed derived from individual self-pollinated progeny derived from a selected F3-F5 plant. The seed sample can be genotyped directly as seed, or as plant tissue grown from such a seed sample. Optionally, seed sharing a common genotype at the specified locus (or loci) are bulked providing a subline that is genetically homogenous at identified loci important for a trait of interest (yield, tolerance, etc.).

An “ancestral line” is a parent line used as a source of genes e.g., for the development of elite lines. An “ancestral population” is a group of ancestors that have contributed the bulk of the genetic variation that was used to develop elite lines. “Descendants” are the progeny of ancestors, and may be separated from their ancestors by many generations of breeding. For example, elite lines are the descendants of their ancestors. A “pedigree structure” defines the relationship between a descendant and each ancestor that gave rise to that descendant. A pedigree structure can span one or more generations, describing relationships between the descendant and it's parents, grand parents, great-grand parents, etc.

An “elite line” or “elite strain” is an agronomically superior line that has resulted from many cycles of breeding and selection for superior agronomic performance. Numerous elite lines are available and known to those of skill in the art of plant breeding. An “elite population” is an assortment of elite individuals or lines that can be used to represent the state of the art in terms of agronomically superior genotypes of a given crop species, such as corn, soybean, or tomato. Similarly, an “elite germplasm” or elite strain of germplasm is an agronomically superior germplasm, typically derived from and/or capable of giving rise to a plant with superior agronomic performance, such as an existing or newly developed elite line of corn, soybean, or tomato.

In contrast, an “exotic strain” or an “exotic germplasm” is a strain or germplasm derived from a plant not belonging to an available elite line or strain of germplasm. In the context of a cross between two soybean plants or strains of germplasm, for example, an exotic germplasm is not closely related by descent to the elite germplasm with which it is crossed. Most commonly, the exotic germplasm is not derived from any known elite line of soybean, but rather is selected to introduce novel genetic elements (typically novel alleles) into a breeding program.

The term “amplifying” in the context of nucleic acid amplification is any process whereby additional copies of a selected nucleic acid (or a transcribed form thereof) are produced. Typical amplification methods include various polymerase based replication methods, including the polymerase chain reaction (PCR), ligase mediated methods such as the ligase chain reaction (LCR) and RNA polymerase based amplification (e.g., by transcription) methods. An “amplicon” is an amplified nucleic acid, e.g., a nucleic acid that is produced by amplifying a template nucleic acid by any available amplification method (e.g., PCR, LCR, transcription, or the like).

A “genomic nucleic acid” is a nucleic acid that corresponds in sequence to a heritable nucleic acid in a cell. Common examples include nuclear genomic DNA and amplicons thereof A genomic nucleic acid is, in some cases, different from a spliced RNA, or a corresponding cDNA, in that the spliced RNA or cDNA is processed, e.g., by the splicing machinery, to remove introns. Genomic nucleic acids optionally comprise non-transcribed (e.g., chromosome structural sequences, promoter regions, enhancer regions, etc.) and/or non-translated sequences (e.g., introns), whereas spliced RNA/cDNA typically do not have non-transcribed sequences or introns. A “template nucleic acid” is a nucleic acid that serves as a template in an amplification reaction (e.g., a polymerase based amplification reaction such as PCR, a ligase mediated amplification reaction such as LCR, a transcription reaction, or the like). A template nucleic acid can be genomic in origin, or alternatively, can be derived from expressed sequences, e.g., a cDNA or an EST.

An “exogenous nucleic acid” is a nucleic acid that is not native to a specified system (e.g., a germplasm, plant, variety, etc.), with respect to sequence, genomic position, or both. As used herein, the terms “exogenous” or “heterologous” as applied to polynucleotides or polypeptides typically refers to molecules that have been artificially supplied to a biological system (e.g., a plant cell, a plant gene, a particular plant species or variety or a plant chromosome under study) and are not native to that particular biological system. The terms can indicate that the relevant material originated from a source other than a naturally occurring source, or can refer to molecules having a non-natural configuration, genetic location or arrangement of parts.

In contrast, for example, a “native” or “endogenous” gene is a gene that does not contain nucleic acid elements encoded by sources other than the chromosome or other genetic element on which it is normally found in nature. An endogenous gene, transcript or polypeptide is encoded by its natural chromosomal locus, and not artificially supplied to the cell.

The term “recombinant” in reference to a nucleic acid or polypeptide indicates that the material (e.g., a recombinant nucleic acid, gene, polynucleotide, polypeptide, etc.) has been altered by human intervention. Generally, the arrangement of parts of a recombinant molecule is not a native configuration, or the primary sequence of the recombinant polynucleotide or polypeptide has in some way been manipulated. The alteration to yield the recombinant material can be performed on the material within or removed from its natural environment or state. For example, a naturally occurring nucleic acid becomes a recombinant nucleic acid if it is altered, or if it is transcribed from DNA which has been altered, by means of human intervention performed within the cell from which it originates. A gene sequence open reading frame is recombinant if that nucleotide sequence has been removed from it natural context and cloned into any type of artificial nucleic acid vector. Protocols and reagents to produce recombinant molecules, especially recombinant nucleic acids, are common and routine in the art. The term recombinant can also refer to an organism that harbors recombinant material, e.g., a plant that comprises a recombinant nucleic acid is considered a recombinant plant. In some embodiments, a recombinant organism is a transgenic organism.

The term “introduced” when referring to translocating a heterologous or exogenous nucleic acid into a cell refers to the incorporation of the nucleic acid into the cell using any methodology. The term encompasses such nucleic acid introduction methods as “transfection,” “transformation” and “transduction.”

As used herein, the term “vector” is used in reference to polynucleotide or other molecules that transfer nucleic acid segment(s) into a cell. The term “vehicle” is sometimes used interchangeably with “vector.” A vector optionally comprises parts which mediate vector maintenance and enable its intended use (e.g., sequences necessary for replication, genes imparting drug or antibiotic resistance, a multiple cloning site, operably linked promoter/enhancer elements which enable the expression of a cloned gene, etc.). Vectors are often derived from plasmids, bacteriophages, or plant or animal viruses. A “cloning vector” or “shuttle vector” or “subcloning vector” contains operably linked parts that facilitate subcloning steps (e.g., a multiple cloning site containing multiple restriction endonuclease sites).

The term “expression vector” as used herein refers to a vector comprising operably linked polynucleotide sequences that facilitate expression of a coding sequence in a particular host organism (e.g., a bacterial expression vector or a plant expression vector). Polynucleotide sequences that facilitate expression in prokaryotes typically include, e.g., a promoter, an operator (optional), and a ribosome binding site, often along with other sequences. Eukaryotic cells can use promoters, enhancers, termination and polyadenylation signals and other sequences that are generally different from those used by prokaryotes.

The term “transgenic plant” refers to a plant that comprises within its cells a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to refer to any cell, cell line, callus, tissue, plant part or plant, the genotype of which has been altered by the presence of heterologous nucleic acid including those transgenic organisms or cells initially so altered, as well as those created by crosses or asexual propagation from the initial transgenic organism or cell. The term “transgenic” as used herein does not encompass the alteration of the genome (chromosomal or extra-chromosomal) by conventional plant breeding methods (e.g., crosses) or by naturally occurring events such as random cross-fertilization, non-recombinant viral infection, non-recombinant bacterial transformation, non-recombinant transposition, or spontaneous mutation.

“Positional cloning” is a cloning procedure in which a target nucleic acid is identified and isolated by its genomic proximity to marker nucleic acid. For example, a genomic nucleic acid clone can include part or all of two more chromosomal regions that are proximal to one another. If a marker can be used to identify the genomic nucleic acid clone from a genomic library, standard methods such as sub-cloning or sequencing can be used to identify and or isolate subsequences of the clone that are located near the marker.

A specified nucleic acid is “derived from” a given nucleic acid when it is constructed using the given nucleic acid's sequence, or when the specified nucleic acid is constructed using the given nucleic acid. For example, a cDNA or EST is derived from an expressed mRNA.

The term “genetic element” or “gene” refers to a heritable sequence of DNA, i.e., a genomic sequence, with functional significance. The term “gene” can also be used to refer to, e.g., a cDNA and/or a mRNA encoded by a genomic sequence, as well as to that genomic sequence.

The term “genotype” is the genetic constitution of an individual (or group of individuals) at one or more genetic loci, as contrasted with the observable trait (the phenotype). Genotype is defined by the allele(s) of one or more known loci that the individual has inherited from its parents. The term genotype can be used to refer to an individual's genetic constitution at a single locus, at multiple loci, or, more generally, the term genotype can be used to refer to an individual's genetic make-up for all the genes in its genome. A “haplotype” is the genotype of an individual at a plurality of genetic loci. Typically, the genetic loci described by a haplotype are physically and genetically linked, i.e., on the same chromosome segment.

The terms “phenotype,” or “phenotypic trait” or “trait” refers to one or more trait of an organism. The phenotype can be observable to the naked eye, or by any other means of evaluation known in the art, e.g., microscopy, biochemical analysis, genomic analysis, an assay for a particular disease resistance, etc. In some cases, a phenotype is directly controlled by a single gene or genetic locus, i.e., a “single gene trait.” In other cases, a phenotype is the result of several genes. A “quantitative trait loci” (QTL) is a genetic domain that is polymorphic and effects a phenotype that can be described in quantitative terms, e.g., height, weight, oil content, days to germination, disease resistance, etc, and, therefore, can be assigned a “phenotypic value” which corresponds to a quantitative value for the phenotypic trait. A QTL can act through a single gene mechanism or by a polygenic mechanism.

A “molecular phenotype” is a phenotype detectable at the level of a population of (one or more) molecules. Such molecules can be nucleic acids such as genomic DNA or RNA, proteins, or metabolites. For example, a molecular phenotype can be an expression profile for one or more gene products, e.g., at a specific stage of plant development, in response to an environmental condition or stress, etc. Expression profiles are typically evaluated at the level of RNA or protein, e.g., on a nucleic acid array or “chip” or using antibodies or other binding proteins.

The term “yield” refers to the productivity per unit area of a particular plant product of commercial value. For example, yield of soybean is commonly measured in bushels of seed per acre or metric tons of seed per hectare per season. Yield is affected by both genetic and environmental factors. “Agronomics,” “agronomic traits,” and “agronomic performance” refer to the traits (and underlying genetic elements) of a given plant variety that contribute to yield over the course of growing season. Individual agronomic traits include emergence vigor, vegetative vigor, stress tolerance, disease resistance or tolerance, herbicide resistance, branching, flowering, seed set, seed size, seed density, standability, threshability and the like. Yield is, therefore, the final culmination of all agronomic traits.

A “set” of markers or probes refers to a collection or group of markers or probes, or the data derived therefrom, used for a common purpose, e.g., identifying soybean plants with a desired trait (e.g., tolerance to pests or drought). Frequently, data corresponding to the markers or probes, or data derived from their use, is stored in an electronic medium. While each of the members of a set possess utility with respect to the specified purpose, individual markers selected from the set as well as subsets including some, but not all of the markers, are also effective in achieving the specified purpose.

A “look up table” is a table that correlates one form of data to another, or one or more forms of data with a predicted outcome that the data is relevant to. For example, a look up table can include a correlation between allele data and a predicted trait that a plant comprising a given allele is likely to display. These tables can be, and typically are, multidimensional, e.g., taking multiple alleles into account simultaneously, and, optionally, taking other factors into account as well, such as genetic background, e.g., in making a trait prediction.

A “computer readable medium” is an information storage media that can be accessed by a computer using an available or custom interface. Examples include memory (e.g., ROM or RAM, flash memory, etc.), optical storage media (e.g., CD-ROM), magnetic storage media (computer hard drives, floppy disks, etc.), punch cards, and many others that are commercially available. Information can be transmitted between a system of interest and the computer, or to or from the computer to or from the computer readable medium for storage or access of stored information. This transmission can be an electrical transmission, or can be made by other available methods, such as an IR link, a wireless connection, or the like.

“System instructions” are instruction sets that can be partially or fully executed by the system. Typically, the instruction sets are present as system software.

Overview of the Basic Processes of the Invention

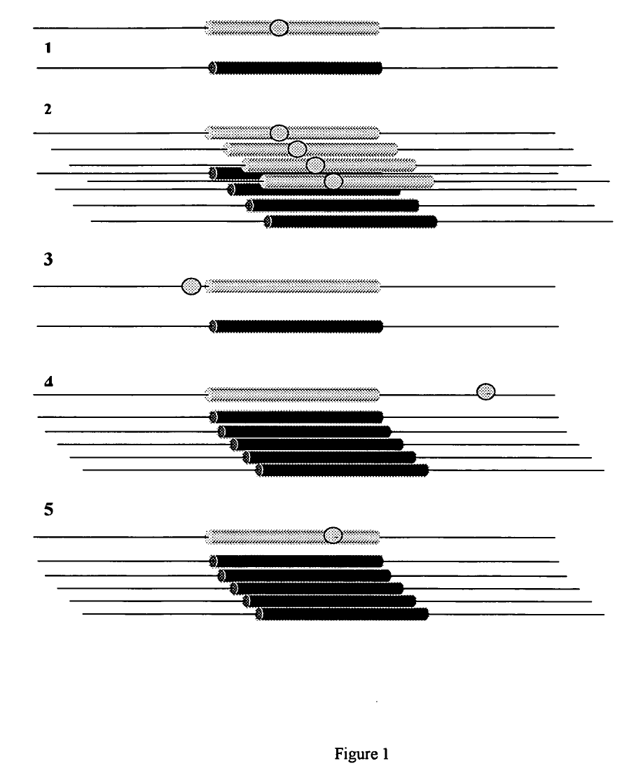

The microarray based analysis of hybridization polymorphisms provides the following possible scenarios (see FIG. 1):

Case 1: Direct detection of the sequence polymorphism within the probe region.

Case 2: Direct detection of the amplified sequence polymorphism within the probe region.

Case 3: Indirect detection of the sequence polymorphism immediately outside the probe region. The sequence polymorphism could form different secondary structures that might affect the hybridization efficiency.

Case 4: Indirect detection of the sequence polymorphism outside the probe region. The sequence polymorphism alters the enzyme restriction site, and thus results in RFLPs. The RFLPs were subsequently preferentially amplified, and this leads to a target abundance difference.

Case 5: Indirect detection of the sequence polymorphism inside the probe region. The sequence polymorphism alters the enzyme restriction site, and thus results in RFLPs. The RFLPs were subsequently preferentially amplified, and this leads to a target abundance difference.

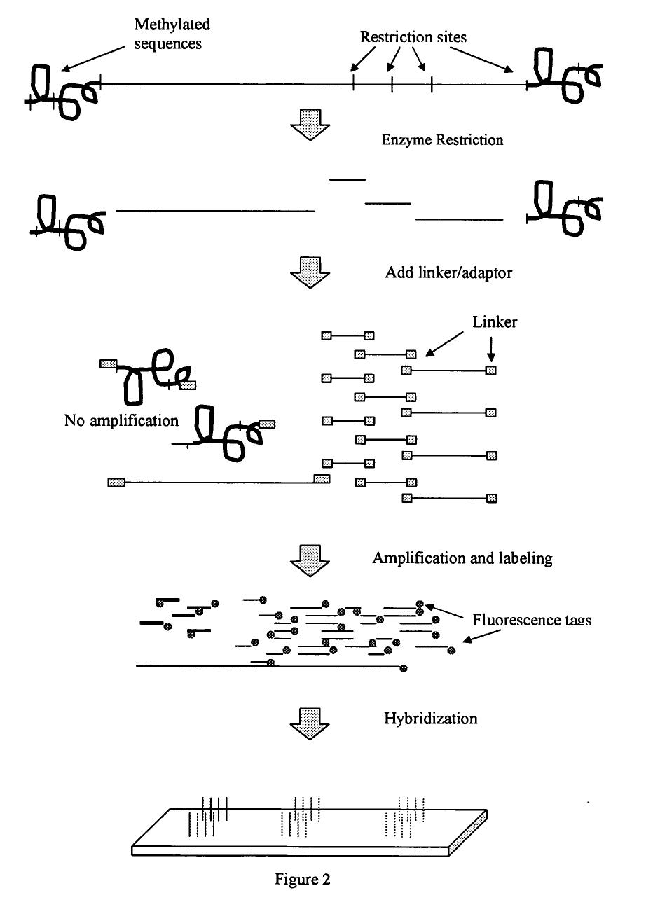

The present method uses the microarray with bound short oligonucleotide probes to indirectly detect the sequence polymorphisms by reading hybridization signal differences (hybridization polymorphisms) in a comparative genomic DNA hybridization experiment. It includes the following major steps (FIG. 2):

A. Select oligonucleotide probes and design a microarray for the detection
1) Select short oligonucleotide sequences (25 mer in the example) that complementary to the genome sequences of interest. Preferably, the probes will be complement the gene sequences (coding or regulatory sequences).
2) The oligonucleotide molecules will be synthesized directly onto the microarray surface or synthesized and deposited onto the microarray surface.
3) The manufactured microarray will determine the coverage of the sequences to be surveyed.

B. Translate Sequence Variations into Variations of Hybridizing Targets (see FIG. 2):

1) Prepare genomic DNA from two genetic varieties using methods of choice Restrict the prepared genomic DNA by site-specific restriction enzymes. As a result, the genomic DNA will be fragmented according to sequence at the restriction site. Sequence variations at the restriction sites will create different length of the restriction fragments (restriction fragment length polymorphisms, or RFLPs). The restriction enzymes used should create several overhang bases. The enzyme used in this step can be a single restriction enzyme or a combination of multiple restriction enzymes. If a methylation sensitive enzyme is used, only the hypomethylation regions will be selectively restricted.

2) This step translates the sequence polymorphism to RFLPs.

3) A pair of DNA oligonucleotides with unique Tm and base composition and partial sequence complementary to the overhang bases of the restriction fragments will be linked to all of the restriction fragments regardless of the fragment size. The universally added oligonucleotides (universal linkers) will be then used as PCR primers for PCR amplification.

4) Under the PCR amplification condition of choice, restriction fragments at the certain range (depend on the extension time used in the PCT amplification) will be selectively amplified. Fragments of large size will not be amplified due to the insufficient extension. By this step, RFLPs will be translated into polymorphisms in target (molecules to be hybridized to the probes) in abundance.

C. Label and Hybridize Targets

1) Amplified targets will be fragmented in a random fashion by DNase or other means into 50-200 base fragments

2) Each short fragment will be end labeled by fluorescence tagged nucleotides of choice unselectively by terminal transferase

3) The labeled target molecules will be used to hybridize to the short oligonucleotide probes on the microarray according to sequence complementation.

4) The fluorescent signal of the labeled target will be captured by the hybridizing probes during hybridization. If a labeled molecule does not have a corresponding probe, it will be washed away. If a labeled molecule is low in abundance, the signal will not be detected by the microarray. This will provide the opportunity to eliminate the noise from large genomic DNA fragments not been fragmented by restriction enzymes, large genomic DNA fragments outside of range for amplification, and fragments without corresponding probes.

D. Quantify Hybridization Signal and Detect Polymorphisms

1) The hybridization signals will be captured by a laser scanner or CCD, and quantified by a computational algorithm.

2) Signals of each probe (feature) obtained from different genetic varieties will be compared. Probes with signal differences will be recorded and statistically analyzed. The origin of the probes with differential signals will be analyzed.

3) The differential signals reflects single nucleotide polymorphism that leads to different binding affinity, amplified restriction fragment polymorphism (RFLP and AFLP) that leads to different target abundance, and sequence polymorphism leads to different secondary structure of the targets.

One example of such detection is illustrated by GSHP detection in maize. In this case, a microarray in the GeneChip format with 1.3 million different oligonulceotide probes is used for the detection. These probes were selected based on the gene coding region sequences. Genomic DNA from Mol7 and B73 is extracted, fragmented using Pst I, a methylation sensitive enzyme, PCR amplified by a pair of universal linkers, labeled with fluorescent tagged nucleotides, and hybridized to the maize GeneChip microarray. Differential signals are detected, recorded and analyzed by statistical methods.

Two alternative labeling methods are described for labeling complex genome that enable the application in economic species which frequently with complex genomes. The target labeling can be achieved using enzyme-mediated end-labeling method (a genome reduction method), or using a random labeling method, in which random hexamer oligonucleotides are used as primers to synthesize Klenow fragments and incorporate the fluorescent tagged nucleotides. Comparing to random labeling, the detection sensitivity and accuracy improved dramatically. Among the polymorphisms selected by the p-value and fold difference, only 1% of the polymorphism detected by random labeling method has the signal difference greater than 5 fold. In contrast, approximately 60% of the polymorphisms detected by the invented methods have the signal difference greater than 5 fold.

Comparing to other genome reduction methods that were previously described, such as high cot DNA, cDNA, or methylation filtration methods, the described method in this invention is unique and has advantages, as shown in Table 1.

TABLE 1
Comparison of invented and previous
published genome reduction methods

| | Genome | | | |
|---|---|---|---|---|
| | fragmenta- | Enrichment of | | |
| | tion | gene fragments | Labeling | Notes |
| Invented | Methylation | Preferential | Regular | High efficiency |
| genome | sensitive | amplification | labeling | in genome reduc- |
| reduction | enzyme | of targets | | tion GC content |
| | restriction | | | dependent, good |
| | Methylation | | | for cereal crops |
| | insensitive | | | Lower conversion |
| | enzyme | | | rate to SNP |
| | restriction | | | |
| Methyl | Methylation | Gel | Regular | Low target |
| filtra- | sensitive | electrophoresis | labeling | recovery and |
| tion | enzyme | separation of | | hybridization |
| | restriction | targets, no | | signals Incon- |
| | | amplification | | sistent target |
| | | | | recovery intro- |
| | | | | duces variation |
| High Cot | Random | Hap | Regular | Inconsistent |
| | shearing, | chromatograhy | labeling | target recovery |
| | de/renature | | | introduces |
| | | | | variation |
| cDNA | | cDNA | Regular | Variations |
| | | | labeling | introduced by |
| | | | | transcription |
| AFLP | Methylation | Preferential | Regular | No gene |
| | insensitive | amplification | labeling | enrichment |
| | enzyme | of targets | | |
| | restriction | | | |

### EXAMPLES

The present method can be used but not limited in following immediate applications which have been widely used in agricultural and medical science and practice: 1) construction of ultra-high density gene map; 2) identify markers for single gene traits or QTLs by bulk segregant analysis (BSA) and similar approaches; 3) associate QTL and candidate genes through whole genome linkage analyses or association studies; and 4) high throughput screening using diagnostic marker.

### Example 1

Materials and Methods

GeneChip Microarray Used for the Assay

A custom designed maize GeneChip microarray (SYNG007) manufactured by Affymetrix was used for the comparative genomic hybridization analysis and maize ultra-high density mapping. The maize GeneChip microarray consists of approximately 1.3 million oligonucleotide probes representing 82,000 unique genes or EST clusters. Only perfect matched probes are included in the array design.

Other arrays described included custom designed tomato GeneChip array, custom designed Arabidopsis whole genome exon array, custom designed Phytophthora array, and commercial Drosophila GeneChip arrays.

Genomic DNA Extraction

Tissue samples are collected from leaf material of two week old seedlings. Genomic DNA (gDNA) was extracted using the CTAB method and the Qiagen DNeasy column (Qiagen). The extracted gDNA were eluted and resuspened in reduced EDTA TE buffer. The quality of the gDNA was determined by gel electrophoresis. The gDNA were quantified using an UV spectrophotometer and adjusted to a final concentration of 250 ng/μl.

Methylation Filtering by Restriction Enzymatic Reaction

The prepared gDNA was digested by the methylation sensitive restriction enzyme PstI. Briefly, 2 μl gDNA (250 ng/μl) was mixed with 2 μl NEB buffer 3, 2 μl BSA, 2 μl PstI and 12 μl nuclease free water in a 20 μl reaction on ice. The contents were mixed by vortexing. The enzyme reaction was carried under the following condition using a thermocycler: 37° C. for two hours, 85° C. for twenty minutes and hold at 4° C.

Ligation of Universal Adaptors

A total of 20 μl PstI digested gDNA was used for the ligation reaction. Two DNA oligonucleotides, with sequence of CAC GAT GGA TCC AGT GCA and CTG GAT CCA TCG TGC A, were used as PstI adaptors. 4 μl of each adaptors were pre-annealed in 2 μl 10× Annealing buffer and 10 μl nuclease free water in 65 oC for 10 min and gradually reduce the temperature to 25° C. over the course of two hours. The ligation reaction contains 2.5 μl NEB T4 DNA Ligase buffer, 1.25 μl PstI Adapter and 1.25 μl NEB T4 DNA Ligase. The reaction was incubated in 16° C. for two hours, terminated by applying 70° C. for twenty minutes and hold at 4° C. Following the reaction, the 25 μl ligation is diluted by adding 75 μl nuclease free water.

PCR Amplification and Purification

Ligated restricted fragments were amplified by polymerase chain reaction (PCR) using PstI adaptors as priming sites. The PCR reaction contains 10 μl diluted ligation reaction, 10 μl PCR buffer, 10 μl dNTP's, 10 μl MgCl₂, 7.5 μl PstI Primer (GAT GGA TCC AGT GCA G), 2.5 μl AmpliTaq Gold polymerase and 50 μl nuclease free water. The amplification was done by using a thermocycler with following program: 95° C. for 3 minutes, 25 cycles of 95° C. for 30 seconds, 59° C. for 30 seconds, and 72° C. for 30 seconds, followed by 72° C. for 7 minutes and hold at 4° C. Fragments with size ranged from 400-1000 bps were amplified, and purified by one of the two methods: by Qiagen QIAquick PCR Purification Kit, or by Qiagen MinElute 96 UF PCR Purification Kit according to manufacturer's instruction. The final concentration of the PCR products was adjusted to a minimum of 450 ng/μl.

Fragmentation and Labeling

PCR products were further fragmented into 50-200 bp fragments in a 55 μl reaction contains 45 μl of purified PCR product equivalent to 20 μg in EB buffer, 5 μl 10× Affymetrix Fragmentation buffer and 5 μl diluted Affymetrix Fragmentation Reagent (DNase I 0.048 U/μl). The reaction was incubated in 37° C. for 30 minutes, 5° C. for fifteen minutes and hold at 4° C.

These small fragments were labeled by in a 70 μl reaction containing 50.6 μl of the fragmented sample and 19.4 μl of the labeling mix at 37° C. for 2 hours and terminated by 95° C. for 15 minutes. The labeling mix consists of 14 μl 5× TdT buffer, 2 μl of GeneChip DNA Labeling Reagent and 3.4 μl Terminal Deoxynucleotidyl Transferase.

Random Labeling of gDNA

The prepared gDNA was denatured in the presence of random octamers. Briefly, 4 μl gDNA (500 ng/μl) was mixed with 20 μl 2.5× random primers solution and 20 μl nuclease free water in a 44 μl reaction on ice. The contents were mixed by vortexing. The reaction was carried out under the following condition using a thermocycler: 99° C. for five minutes and hold at 4° C.

To the 44 μl sample the following additions were made on ice; 5 μl biotin labeled dNTP mixture and 1 μl Klenow Fragment. The contents were mixed by vortexing. The reaction was carried out under the following condition using a thermocycler: 37° C. for two hours and hold at 4° C.

Hybridization, Washing, Staining and Scanning

The labeled PCR fragments (targets) were hybridized to the oligonucleotide probes on the GeneChip microarray. Briefly, prehybridize GeneChip microarrays 200 μl 1× hybridization buffer at 42° C. for 10 minutes using an Affymetrix hybridization oven at 60 RPM. A 250 μl reaction containing70 μl labeled gDNA, 2.5 μl B2 control oligo, 2.5 μl 100× RNA control, 2.5 μl Herring Sperm DNA, 2.5 μl Acetylated BSA, 125 μl 2× Hybridization buffer, 18.75 μl DMSO, 22.25 μl DEPC water and 4 μl Affymetrix Reagent X were pre-incubated at 99° C. for 5 minutes and 42° C. for 5 minutes. The pre-treated hybridization cocktail was then applied to the GeneChip array and hybridize in the hybridization oven at 42° C. with 60 RPM for 16 hours. Following hybridization, the GeneChip arrays were washed and stained using the fluidic protocol EukGE-WS2v4_{—}450 according to Affymetrix instructions. The images of the arrays were acquired using an Affymetrix GeneChip Scanner-3000. Image data were processed using Affymetrix GCOS program.

### Example 2

Construction of an Ultra High Density Linkage Map in Maize

A custom designed maize GeneChip microarray was developed to identify single feature polymorphism (SFP) within coding sequences at a genome scale. This GeneChip microarray has 1.3 million 25 mer oligonucleotide probes for approximately 82,000 genes and EST clusters. Approximately 14400 SFPs, representing 1% of the total number of the screened features, were identified between B73 and M017. Using these hybridization polymorphisms as markers, a maize ultra high-density map was developed for the intermated B73 and Mol7 population (IBM). 4368 gene markers were mapped by 10997 SFPs. Ninety-three percent of the studied SFPs can be validated by the segregation pattern of the associated known RFLP fragments. Further sequence analysis of these SFPs confirmed the associated single nucleotide polymorphisms (SNPs) in the probe regions. Using a pattern match method, we further mapped 34,034 SFPs representing 11,427 unique genes or EST clusters. The mapped genes are validated by sequence analyses, and supported by the macro synteny relations between rice and maize. Integration of these gene markers with other types of genetic and physical markers will facilitate marker assisted breeding and identification of genes that controls complex traits. See Appendix for detailed methods.

### Example 3

Construction of an Ultra High Density Bin Map in Tomato

A total of 74 introgression lines (ILs) and their parents were screened for hybridization polymorphisms by comparative genomic hybridization using a custom designed tomato GeneChip microarrays. Because they are detected by single DNA oligonucleolide probes (features) representing the gene fragments, the hybridization signal differences are named single feature polymorphisms (SFPs).

The DNA hybridization for the two parental lines was replicated 8 times to ensure the statistical significance of the detection. Although no replication for DNA hybridization of most of the ILs, hybridization experiments for selected ILs associated with important traits were replicated twice and can be done more to increase the number of markers associated with these traits.

Data quality was first evaluated using Refiner, a data quality module in Expressionist (GeneData). All data met the median to high quality standard. Reproducibility of the DNA hybridization experiments was examined and an average correlation coefficient of 0.93 was achieved across all 18 parental line data sets. Approximately 8364 SFPs between the two parental lines were identified using two independent statistical methods with a set of high stringency statistical criteria. The false discovery rate of these SFPs was estimated to be less than 0.1%. The statistical methods used were validated by the cross validation method. In average the genotype of a tester can be correctly assigned 97% of the time based on the identified SFPs. The identified SFPs were validated molecularly and genetically. A total of 131 gene fragments containing SFPs were PCR amplified, and sequenced. Among them, 101 were confirmed and led to a confirmation rate of 77%.

Probes used to detecting SFPs were associated with 375 known genetic markers by aligning sequence of DNA oligonucleotide probe sets with marker sequences. 82 markers were found to be overlapped by 8364 probes detecting SFPs. By comparing the hybridization signal of each locus (feature) in the ILs to the parental reference, allele of each locus in the ILs was assigned. Allele assignment based on marker associated SFPs was compared to the allele assignment from the genetic study. 90% of the 6560 genotypes were assigned in agreement with the allelic information from the previous mapping study. From these 8364 SFPs, 1630 high-confidence gene markers were identified and mapped to the genetic bins. Approximately 70-90 SFP markers are being selected and validated. Other SFPs were studied computationally and molecularly to refine our approach and seek for additional SFP markers.

### Example 4

Identification of Trait Markers in Tomato

A bulked segregant analysis approach was used to identify close linked markers for the Fusarium resistance locus Fr1. Two pools of genomic DNA were prepared from an F2 population, one from 22 individuals homozygous for the resistant allele and the other from 21 individuals homozygous for the susceptible allele. The two pools of genomic DNA and genomic DNA from the parental lines were prepared, labeled and each hybridized to the custom designed tomato GeneChip microarrays according to the procedures described in Appendix. Probes detecting differential hybridization signals (p<0.001, fold difference>1.5) between the two pools were selected as candidate markers. The candidates were further sequenced to identify SNPs and then mapped to determine linkage to the target locus. 16 of 17 candidate markers identified in this experiment were found to be genetically linked to the target locus by scoring 43 individual lines that made up the resistant and susceptible BSA pools. Further testing of 90 well characterized resistant and susceptible tomato varieties also confirmed the tight linkage, and validated the robustness of the approach. Similar approach has identified closely linked genetic markers for Stemphillium resistance locus Sm and Phytophthora resistance loci.

### Example 5

Identification of SFPs in Closely Related Species Using Heterologous Detection

Genomic DNA was extracted from different varieties of pepper, labeled by the random heximer method, and hybridized to the custom designed tomato GeneChip microarray according to the procedures described in Appendix. The experiments were replicated ten times for each variety. The two species belongs to Solanaceae, and share 90% of similarity in coding sequences at the nucleotide level. Under a stringent hybridization condition as described, approximately N % of the tomato probes detected pepper target signals. A total of 1248 putative SFPs between C (chinense P1159234) and F (frutescens, BG2814-6) parents was detected using the criteria of fold difference>1.5 and p<0.01.

Among those, 137 SFPs have aligned pepper sequences (with average 5 bases differences), and 60 of them were selected for validation of SNPs. The results showed that 40-60% of the SFPs are caused by the SNPs within or adjacent to the 25 mer probes.

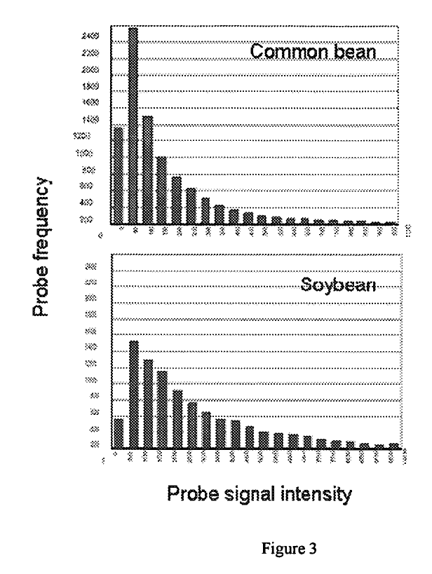

Similarly, SFPs were successfully detected in Brassica and sugar beets using Arabidopsis GeneChip arrays, in leafminer using Drosophila GeneChip arrays, in Plasmopara viticola using Phytophthora GeneChip arrays, and in common bean using soybean arrays (see FIG. 3).

### Example 6

Identification of SFPs Between Maize Parents B73 and Mol7or Different Genetic Lines

Six to ten replicate genomic hybridization data sets each from B73 and Mol7 or genetic lines from other species were used for data analysis. A custom perl script was developed and used to identify the SFPs between these two parents. Briefly, the intensity signals for each hybridization were normalized to the mean value of all features on the chip after they were loaded into the program. The normalized intensities were natural log transformed, and value difference of each feature between parents is subject to t-test. The significant calls were then filtered with fold change criteria. In order to generate data outputs under different stringency, different p-value and fold change cut offs were used. The predicted candidates with significant differences under the defined criteria were selected and used as single feature polymorphism (SFP). These candidates were further validated computationally or experimentally by sequencing.

### Example 7

Cross Validations of Identified SFP

The SFPs predicted based on multiple t-tests at the feature level were further examined by cross validations. In this analysis, one of replicates was removed from the data set as the tester. The new data set was used to identify SFPs between the parents based on t-test. By comparing to newly produced SFP data, the identity of the tester could be assigned. Because the tester was selected from one of two parental lines, the assigned identity is expected to agree with the original identity. Total of 18 cross validations were carried out from different combinations of one replicate left, and the average of the agreed rates was computed.

### Example 8

Genotype Assignments in the Progeny Lines

The genotypes of all 93 progeny lines for any giving SFP were determined using the following algorithm. For each identified SFP, the intensity signals on all parental line replicates were considered to follow two normal distributions, one from B73, and another from Mol7. Each distribution curve shape could be determined by the mean and the standard deviation values for the giving feature in a parental line, which were generated in the SFP identification process. Because of this intermated population has been self-crossed for 6-7 generations, it is believed that the frequency of heterozygous genotype is very low. Hence, only homozygous genotype was considered in the computation. In order to assign the genotype based on the quantitative intensity measurement, the intensity value was first normalized and log transformed as described above. Then the value was used for the area computations: for the distribution with the smaller mean (left), an integration from the negative far away to this value covered by the distribution was computed; while for the distribution with the bigger mean (right), an integration from this value to the positive far away covered by the distribution was computed, as described in follows:

$${A}_{left} = {\int }_{- ∞}^{{x}_{g}} \frac{1}{{σ}_{1} \sqrt{2 π}} {ⅇ}^{- {( x - {μ}_{1} )}^{2} / ( 2 {σ}_{1}^{2} )} ⅆ x$$

$${A}_{right} = {\int }_{{x}_{g}}^{+ ∞} \frac{1}{{σ}_{2} \sqrt{2 π}} {ⅇ}^{- {( x - {μ}_{2} )}^{2} / ( 2 {σ}_{2}^{2} )} ⅆ x$$

A_{left} and A_{right} are the areas described above; xg is the giving intensity after normalization and log transformation; μ1 and μ2 are the means of left and right distributions; σ1 and σ2 are the standard deviations of left and right distributions. These two areas were then compared, and the distribution with the smaller computed area was assigned to the giving intensity.

### Example 9

Validation of Genotype Assignments

Segregation patterns of 1343 previously published genetic markers in the IBM population were used as reference to validate the assigned genotypes (Lee et al. 2002). The genotypic information was downloaded from www.maizegdb.org. The sequences of these genetic markers were blasted against maize probe set sequences to establish the links between the genetic markers and SFP gene markers. The SFP genotypes across all progeny lines with those maize probe set ids were retrieved from the genotype assignment data set, and were compared to the corresponding genetic marker data. The percentage of agreed genotypes was then computed.

### Example 10

Marker Condensations: from SFP to Probe Set Marker

Multiple SFPs from the same probe set were used to evaluate the confidence level of the gene markers. This is accomplished by first searching multiple polymorphic features within a probe set, and assumes that there is no recombination occurred among them. These SFPs were compared within the probe set, and the most frequent genotype was selected as representative for the probe set. For those probe sets with equal number of different genotypes at the feature level, they are ignored as missing data from the calculation.

### Example 11

Genetic Mapping Using the Modified MapMaker Program

Three different genotype data sets were used in the mapping analysis: a) public RFLP and SSR markers; b) Syngenta SSR markers; and c) probe set level markers as described above. The original MapMaker (Lander et al. 1987) was modified in to a UNIX command line program mapmaker500 to accommodate the large number of SFP markers (Yiping Fan et al, Syngenta, unpublished). The computations were carried out using MapMaker500. The 1127 public markers were used as the anchors to generate a framework. The markers from other data sets were then assigned to the appropriate chromosomes based on the best LOD scores to the anchors in the framework. The marker locations were then determined using “build” command. To minimize the impact from random effect, five independent runs were carried out and the common order was selected as the genetic order. For those markers that could be mapped in the multiple locations for a giving LOD difference cut off, the most stringent LOD cut off was used until it was mapped into a single location. In the probe set markers, the data was split into different subgroups based on the stringency used on the SFP identifications and whether a giving marker came from multiple SFPs. The markers in the group with the most stringent conditions were involved in mapping computation first. The mapped markers as well as the anchors were then formed new anchor framework, and the markers in the group with the second most stringent conditions were involved in the computation. The process was repeated until all group markers computed.

### Example 12

Comparison of SFP Gene Map and Previous Genetic Map

In order to evaluate the quality of the map we generated, the SFP gene map and IBM2 map (www.maizegdb.org) were compared. This map combined the genetic association map as well as the physical map; hence the markers on the map could come from different source. To do this comparison, the marker sequences on the public map were blasted against maize probe set sequences, and links between public marker ids and probe set ids were generated. The overlapped markers in both maps were then identified, and the locations were compared.

### Example 13

Maize Map Expansion Using Pattern Matching Algorithm (PMA)

For each mapped genetic marker, the genotypes across all mapping lines were retrieved and the corresponded hybridization .cel files were separated into two bulks, based on their genotypes. This mapped marker was used as “bait”. The t-tests were applied between those two bulks on the feature levels after the intensities were normalized and log transformed. The significant calls were then filtered with the fold change criteria as described above. The output data was parsed as follow. First, the p-values were transformed to the “score” using negative log with base 10 for the computational convenience. Then, for each significant call, if it was not on the current map, its identity and the scores in all baits were collected. The bait with the highest score was then identified and selected. Next, enforce the significant calls to get genotype data as described above, and deleted the calls if the genotypes had more than a number of differences compared to the genotypes in the highest score bait. Finally, for those probe sets with multiple significant calls, the condensation process was used: the best location for this probe set should be the region with the baits that majority of significant features pointed to.

### Example 14

Confirmation of PstI RFLP

The sequences with SNP information between B73 and Mol7 were extracted from maize sequence and SNP dataset. All SNPs and the flanking sequences were searched for the restriction enzyme PstI recognizing sequence CTGCAG. The sequences with PstI polymorphisms were then blasted against maize genomic sequences to find longer sequences that covered the polymorphic PstI sites. Those longer genomic sequences were blasted against maize probe set and individual feature sequences. For those probe sets that were found located around the polymorphic PstI sites, their individual feature behaviors, including the intensities, fold changes, and p-values in the t-tests were extracted from all feature behaviors dataset. The sequences and corresponded feature behaviors were then compared and analyzed to conclude whether the PstI RFLP could be confirmed.

### REFERENCES

Lander E S, Green P, Abrahamson J, Barlow A, Daly M J, Lincoln S E, Newburg L. (1987). MAPMAKER: an interactive computer package for constructing primary genetic linkage maps of experimental and natural populations. Genomics. 1:174-181.

Lee M, Sharopova N, Beavis W D, Grant D, Katt M, Blair D, Hallauer A. (2002). Expanding the genetic map of maize with the intermated B73×Mol7 (IBM) population. Plant Mol Biol. 48:453-461.

What is claimed is:

1. A method for the screening of genomic nucleic acid material for gene specific hybridization polymorphisms, the method comprising: a) global screening for hybridization polymorphisms using microarray; b) enzyme mediated genome complexity reduction; c) enzyme mediated differential signal amplification and noise reduction; d) data extraction and GSHP identification; and e) use of GSHPs in high throughput screening.

2. A method for the screening of genomic nucleic acid material for gene specific hybridization polymorphisms, the method comprising:
a. selecting oligonucleotide probes and designing a microarray comprising said probes for the detection of sequence variation;
b. translating sequence variations into variations of hybridizing targets;
c. labeling and hybridizing the targets;
d. detecting a hybridization signal; and
e. quantifying the hybridization signal and detecting polymorphisms.

3. A method for detection of gene specific hybridization polymorphisms in polynucleotide sequences of genomic DNA, the method comprising:
a. selecting short oligonucleotide sequences complementary to the genomic polynucleotide sequences, said short oligonucleotide sequences to be synthesized directly onto or synthesized and placed onto a microarray surface;
b. preparing genomic DNA from two genetic sources and subjecting said genomic DNA to site-specific restriction using one or more restriction enzymes to produce restriction fragment length polymorphisms (RFLPs);
c. selectively amplifying RFLPs of a selected size range to create amplified polymorphism targets;
d. fragmenting the amplified targets randomly into fragments of from about 50 to about 200 bases and end-labeling the fragments unselectively;
e. hybridizing the end-labeled fragments to the short oligonucleotide sequences on the microarray surface; and
f. quantifying the signals from the hybridization and detecting polymorphisms.

4. The method of claim 3 wherein the short oligonucleotides selected in step a. are from about 25 mers to about 30 mers.

5. The method of claim 3 wherein the fragments of the amplified targets of step d. are end-labeled using fluorescence-tagged nucleotides and a terminal transferase.

6. The method of claim 3 wherein the hybridization signals of step f. are captured by a device selected from the group consisting of a laser scanner and a CCD.

7. The method of claim 6 wherein the captured hybridization signals are quantified using a computational algorithm.

8. The method of claim 3 further comprising: g. comparing signals from different genetic backgrounds or varieties for signal differences and determining the origins of the differential signals.

9. The method of claim 8 further comprising: h. identifying the single nucleotide polymorphisms that cause the differential signals of step g.

10. The single nucleotide polymorphisms identified in the method of claim 9.

11. A genetic map developed using the information generated in the method of claim 3.

12. A genetic map developed using the information generated in the method of claim 8.

13. Molecular markers developed using the information generated in the method of claim 3.

14. Molecular markers developed using the information generated in the method of claim 8.

15. A quantitative trait locus identified and defined using the information generated in the method of claim 3.

16. The quantitative trait locus of claim 15 further characterized using the molecular markers of claim 13.

17. The quantitative trait locus of claim 15 further characterized using the molecular markers of claim 14.
